(19) United States
(12) Patent Application Publication
(10) Pub. No.: US 2019/0247017 A1
(43) Pub. Date: Aug. 15, 2019

(54) INNER MEMBER FOR INTRAVASCULAR IMAGING DEVICE AND ASSOCIATED DEVICES, SYSTEMS, AND METHODS

(71) Applicant: KONINKLIJKE PHILIPS N.V., EINDHOVEN (NL)

(72) Inventors: Maritess Minas, San Diego, CA (US); Jeremy Stigall, Carlsbad, CA (US); Princeton Saroha, Ladera Ranch, CA (US); David Kenneth Wrolstad, Fallbrook, CA (US)

(21) Appl. No.: 16/343,223

(22) PCT Filed: Oct. 19, 2017
(86) PCT No.: PCT/EP2017/076657

§ 371 Date: Apr. 18, 2019

Related U.S. Application Data
(60) Provisional application No. 62/413,631, filed on Oct. 27, 2016.

(51) Int. Cl. A61B 8/12; A61B 8/06; A61B 8/08; A61B 8/00

(52) U.S. Cl. CPC A61B 8/12; A61B 8/06; A61B 8/4494; A61B 8/445; A61B 8/0891

(57) ABSTRACT

An intravascular imaging device is provided. In one embodiment, the intravascular device includes a flexible elongate member sized and shaped for insertion into a vessel of a patient, the flexible elongate member having a distal portion and a proximal portion; and a physiologic sensor assembly disposed at the distal portion, wherein the distal portion of the flexible elongate member comprises a distal inner member coupled to the physiologic sensor assembly, the distal inner member including a first groove extending longitudinally along the distal inner member, and wherein one of a stiffening wire extending longitudinally within the flexible elongate member or an electrical cable coupled to the physiologic sensor assembly is positioned within the first groove of the distal inner member.

### TECHNICAL FIELD

The present disclosure relates generally to intravascular ultrasound (IVUS) imaging and, in particular, to the structure of an intravascular imaging device. For example, the structure at a distal portion can include a distal inner member coupled to an imaging assembly. The distal inner member can be structured to allow the intravascular imaging device to be easily moved through and manipulated within vessels of a patient's body.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

There are two types of IVUS catheters commonly in use today: rotational and solid-state. For a typical rotational IVUS catheter, a single ultrasound transducer element is located at the tip of a flexible driveshaft that spins inside a plastic sheath inserted into the vessel of interest. The transducer element is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer.

Solid-state IVUS catheters carry a sensing assembly or scanner assembly that includes an array of ultrasound transducers distributed around its circumference along with one or more integrated circuit controller chips mounted adjacent to the transducer array. The solid-state IVUS catheters are also referred to as phased array IVUS transducers. The controllers select individual transducer elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

Manufacturing an intravascular imaging device that can efficiently access coronary anatomy and tortuous vascular regions within a human body is challenging. For example, some phased array IVUS transducers have low torsional strength and torque quality. This prevents a physician from easily moving the intravascular device through the patient's vessel, and adjusting the position and/or orientation of the intravascular imaging device as needed. Thus, there is a need to improve torsional strength and torque quality of intravascular imaging devices. Reducing the outer profile or diameter/size of intravascular imaging devices also allows the device to more efficiently traverse vasculature.

### SUMMARY

Embodiments of the present disclosure provide an improved intravascular sensing device for obtaining one or more types of physiological data within vessels in a patient's body, such as intravascular images, flow data, pressure data, etc. Multiple, flexible, tubular components can be joined together to formed the device. For example, a distal portion of the intravascular sensing device can include a distal inner member coupled to a physiological sensor. The distal inner member extends along a length of the distal portion. The distal inner member includes one or more surface grooves extending along at least a portion of the length. The surface grooves are sized and shaped to allow a stiffening wire to be secured or fitted into one surface groove and an electrical cable to be secured or fitted into another surface grove. The stiffening wire improves the responsiveness the intravascular device when a physician moves and turns the intravascular sensing device to steer the device through the patient's vessel. Fitting of the stiffening wire and the electrical cable in the surface grooves allow for a low-profile or compact intravascular sensing device, which can travel through the patient's vessels more efficiently.

In one embodiment, an intravascular imaging device is provided. The intravascular device includes a flexible elongate member sized and shaped for insertion into a vessel of a patient, the flexible elongate member having a distal portion and a proximal portion; and a physiologic sensor assembly disposed at the distal portion, wherein the distal portion of the flexible elongate member comprises a distal inner member coupled to the physiologic sensor assembly, the distal inner member including a first groove extending longitudinally along the distal inner member, and wherein one of a stiffening wire extending longitudinally within the flexible elongate member or an electrical cable coupled to the physiologic sensor assembly is positioned within the first groove of the distal inner member.

In some embodiments, the distal inner member is tubular in shape, and wherein the first groove extends longitudinally along an outer surface of the distal inner member, and/or wherein the distal inner member includes a second groove extending longitudinally along the outer surface, and wherein the other of the stiffening wire or the electrical cable is positioned within the second groove of the distal inner member, and/or wherein the first groove and the second groove are circumferentially spaced from one another, and/or wherein the distal inner member includes a first distal end; a first proximal end; a first inner member portion at the first distal end and coupled to the physiologic sensor assembly; and a second inner member portion adjacent to the first inner member portion, and/or wherein the first groove extends longitudinally along a length of an outer surface of the second inner member portion, and/or wherein the flexible elongate member comprises a proximal outer member disposed at the proximal portion of the flexible elongate member; and a distal outer member disposed at the distal portion of the flexible elongate member, wherein the distal outer member includes a second proximal end coupled to the proximal outer member and a second distal end coupled to the physiologic sensor assembly, wherein the distal inner member extends longitudinally through the distal outer member, and wherein the stiffening wire is coupled to the proximal outer member, and/or wherein the proximal outer member is tubular in shape, and wherein the stiffening wire includes a third proximal end attached to an inner surface of the proximal outer member; and a third distal end positioned within the first groove of the distal inner member, and/or wherein the distal inner member includes a third inner member portion adjacent to the second inner member portion, and wherein the third inner member portion defines a guide wire exit port disposed at a coupling junction between the proximal outer member and the distal outer member, and/or wherein the physiologic sensor assembly comprises an array of intravascular ultrasound (IVUS) transducers disposed on a flex circuit positioned circumferentially around a support member, and wherein the first inner member portion extends through a lumen of the support member and distally beyond the support member, and/or wherein the distal inner member is tubular in shape, and wherein the second inner member portion has a greater outer diameter than the first inner member portion, and/or further comprising a distal tip member coupled to the first distal end of the distal inner member.

In one embodiment, a method of assembling an intravascular device is provided. The method includes obtaining a first flexible elongate member including a first groove and a second groove extending longitudinally along the first flexible elongate member; positioning a stiffening wire in the first groove, wherein the stiffening wire is coupled to a second flexible elongate member; and positioning an electrical cable in the second groove, wherein the electrical cable is coupled to a physiologic sensor assembly.

In some embodiments, the first flexible elongate member is tubular in shape, and wherein the first groove and the second groove extend longitudinally along an outer surface of the first flexible elongate member, and/or wherein the obtaining includes molding the first flexible elongate member to integrally form the first groove and the second groove on the outer surface of the first flexible elongate member, and/or wherein the obtaining includes removing material from a blank of the first flexible elongate member to form the first groove and the second groove on the outer surface of the first flexible elongate member, and/or wherein the first flexible elongate member includes a first distal end; a first proximal end; a first inner member portion at the first distal end; and a second inner member portion adjacent to the first inner member portion, wherein the method further comprises positioning the first flexible elongate member in a first lumen of a third flexible elongate member sized and shaped for insertion into a vessel of a patient, wherein the third flexible elongate member includes a second distal end and a second proximal end, wherein the first flexible elongate member is positioned along a longitudinal axis of the third flexible elongate member such that the first inner member portion extends beyond the second distal end of the third flexible elongate member, and wherein the first flexible elongate member is a distal inner member, the second flexible elongate member is a proximal outer member, and the third flexible elongate member is a distal outer member, and/or further comprising reducing an outer diameter of the first inner member portion based on an inner diameter of a second lumen of the physiologic sensor assembly; and coupling the first flexible elongate member to the physiologic sensor assembly by positioning the first flexible elongate member within the physiologic sensor assembly such that the first inner member portion extends through the second lumen of the physiologic sensor assembly and distally beyond the physiologic sensor assembly, and/or further comprising coupling a distal tip member to the first distal end of the first flexible elongate member after coupling the first flexible elongate member to the physiologic sensor assembly, and/or wherein the first flexible elongate member further includes a third inner member portion adjacent to the second inner member portion, and wherein the method further comprises coupling the second flexible elongate member to the second proximal end of the third flexible elongate member; and creating a guide wire exit port at a coupling junction between the second flexible elongate member and the third flexible elongate member during the coupling such that the third inner member portion is in communication with the guide wire exit port.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of a physiologic sensing system, according to aspects of the present disclosure.

FIG. 2 is a diagrammatic top view of a portion of physiologic sensor assembly in a flat configuration, according to aspects of the present disclosure.

FIG. 3 is a diagrammatic side view of physiologic sensor assembly, including a flex circuit in a rolled configuration around a support member, according to aspects of the present disclosure.

FIG. 4 is a diagrammatic cross-sectional side view of an intravascular device, according to aspects of the present disclosure.

FIG. 5 is a diagrammatic perspective view of a distal inner member, according to aspects of the present disclosure.

FIG. 6 is a diagrammatic cross-sectional view of a distal inner member, according to aspects of the present disclosure.

FIG. 7 is a diagrammatic perspective view of a distal inner member, at least a portion of which is sized and shaped to fit into an inner diameter of a physiologic sensor assembly, according to aspects of the present disclosure.

FIG. 8 is a diagrammatic perspective view of a distal inner member arranged to be positioned within a distal outer member, according to aspects of the present disclosure.

FIG. 9 is a diagrammatic perspective view of a distal inner member positioned in a distal outer member, according to aspects of the present disclosure.

FIG. 10 is a diagrammatic perspective view of a distal inner member bonded to a physiologic sensor assembly, according to aspects of the present disclosure.

FIG. 11 is a diagrammatic perspective view of an electrical cable of a physiologic sensor assembly positioned in a groove of a distal inner member, according to aspects of the present disclosure.

FIG. 12 is a diagrammatic perspective view of a stiffening wire positioned for alignment into a groove of a distal inner member, according to aspects of the present disclosure.

FIG. 13A is a diagrammatic perspective view of a proximal outer member assembled with a distal outer member, according to aspects of the present disclosure.

FIG. 13B is a diagrammatic cross-sectional view of a distal outer member, a distal inner member, an electrical wire, and a stiffening wire coupled in position, according to aspects of the present disclosure.

FIG. 14 is a diagrammatic perspective view of a distal tip member bonded to a distal inner member, according to aspects of the present disclosure.

FIG. 15 is a flow diagram of a method of assembling an intravascular sensing device, according to aspects of the present disclosure.

FIG. 16 is a flow diagram of a method of assembling an intravascular sensing device, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The size and torque quality of an intravascular device can impact the deliverability of the intravascular device for catheterization procedures. For example, an intravascular device with a lower profile or more compact size may allow for access to more restricted vascular regions, such as those vessel with a smaller diameter and/or vessels with tortuous anatomy. A higher torque quality may improve rotational, navigational, and/or directional capabilities. Disclosed herein are various embodiments for providing an improved intravascular device. The disclosed intravascular device includes a distal inner member including surface grooves for fitting cables and/or wires that extend through the intravascular device. The distal inner member is disposed at a distal portion of the intravascular device. More distal portions of the intravascular device are positioned farther within a patient body. The fitting of cables and/or wires within the surface grooves maximizes space utilization within the intravascular device and thus enables a more compact-sized or lower-profiled intravascular device. The disclosed intravascular device further includes fitting a stiffening wire in one of the surface grooves and extending the distal inner member to a distal-most tip of the intravascular device to improve torque performance. For example, the distal portion of the intravascular device, within the patient body, can respond can rotate, translate, and/or otherwise move better when corresponding movement is applied by a physician to a proximal portion of the device, outside the patient body. Although the disclosed embodiments are described in the context of intravascular ultrasound (IVUS) imaging devices, the disclosed embodiments are suitable for use in any type of physiologic sensing device.

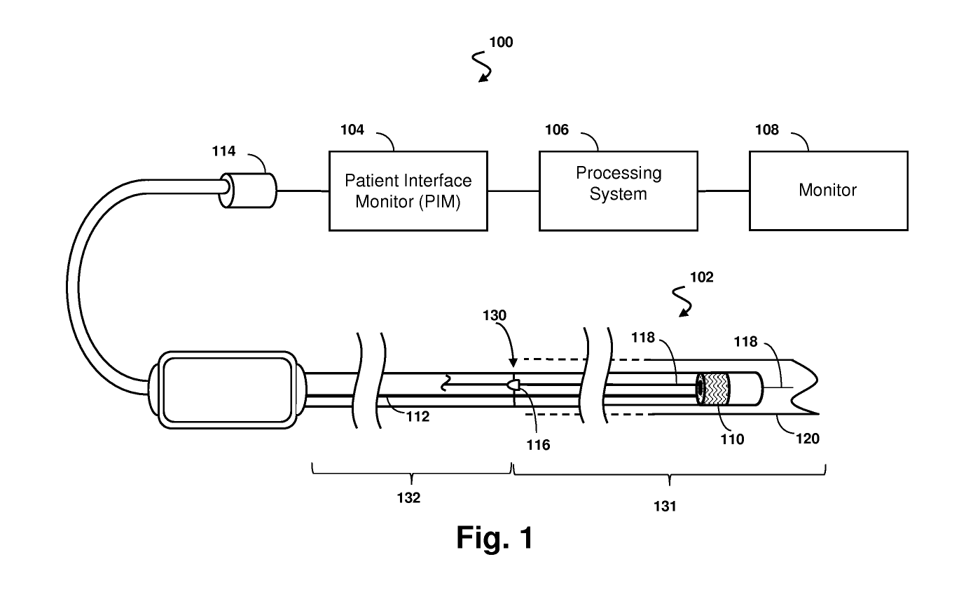

FIG. 1 is a diagrammatic schematic view of a physiologic sensing system 100, according to aspects of the present disclosure. The system 100 may include an intravascular device 102 such as a catheter such as a catheter, guide wire, or guide catheter, a patient interface module (PIM) 104, a processing system 106, such as a console and/or a computer, and a monitor 108.

In some embodiments, the intravascular device 102 may be an imaging device, such as an IVUS imaging device. The intravascular device 102 may include a physiological sensor assembly 110 mounted at a distal portion 131 near a distal end of the intravascular device 102. In instances in which the intravascular device 102 is an IVUS device, the physiological sensor assembly 110 is an imaging assembly, such as an IVUS imaging assembly. At a high level, the intravascular device 102 emits ultrasonic energy from a transducer array included in physiologic sensor assembly 110. The ultrasonic energy is reflected by tissue structures in the medium, such as a vessel 120, surrounding the physiologic sensor assembly 110, and the ultrasound echo signals are received by the transducer array in the physiologic sensor assembly 110. Although the physiologic sensor assembly 110 is illustrated with a configuration for a transducer array, the physiologic sensor assembly 110 may be alternatively configured to include a rotational transducer to achieve similar functionalities. The PIM 104 transfers the received echo signals to the processing system 106 where the ultrasound image (including the flow information) is reconstructed and displayed on the monitor 108. The processing system 106 can include a processor and a memory. The processing system 106 can be operable to facilitate the features of the system 100 described herein. For example, the processor can execute computer readable instructions stored on the non-transitory tangible computer readable medium.

In other embodiments, the intravascular device 102 can include scanner assembly, imaging assembly, or any suitable type of physiologic sensing assembly configured to obtain physiologic data associated with pressure, flow, temperature, forward looking IVUS (FL-IVUS), intravascular photoacoustic (IVPA) imaging, a fractional flow reserve (FFR) determination, a functional measurement determination, a coronary flow reserve (CFR) determination, optical coherence tomography (OCT), computed tomography, intracardiac echocardiography (ICE), forward-looking ICE (FLICE), intravascular palpography, transesophageal ultrasound, and/or other suitable types of physiologic data.

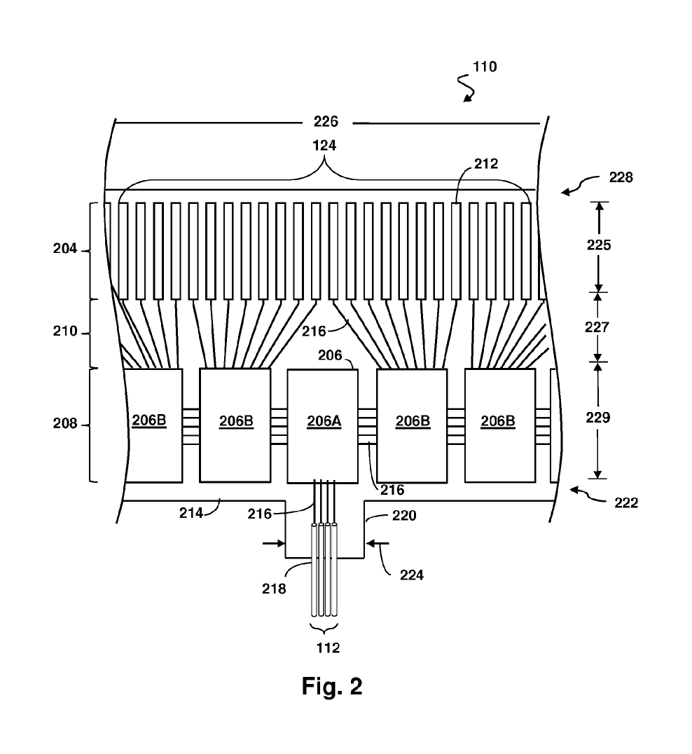

The PIM 104 facilitates communication of signals between the processing system 106 and the physiologic sensor assembly 110 included in the intravascular device 102. This communication includes the steps of: (1) providing commands to integrated circuit controller chip(s) 206A, 206B, illustrated in FIG. 2, included in the physiologic sensor assembly 110 to select the particular transducer array element(s) to be used for transmit and receive, (2) providing the transmit trigger signals to the integrated circuit controller chip(s) 206A, 206B included in the physiologic sensor assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) 206 of the physiologic sensor assembly 110. In some embodiments, the PIM 104 performs preliminary processing of the echo data prior to relaying the data to the processing system 106. In examples of such embodiments, the PIM 104 performs amplification, filtering, and/or aggregating of the data. In an embodiment, the PIM 104 also supplies high- and low-voltage direct current (DC) power to support operation of the device 102 including circuitry within the physiologic sensor assembly 110.

The processing system 106 receives the echo data from the physiologic sensor assembly 110 by way of the PIM 104 and processes the data to reconstruct an image of the tissue structures in the medium surrounding the physiologic sensor assembly 110. The processing system 106 outputs image data such that an image of a vessel, such as a cross-sectional image of the vessel 120, is displayed on the monitor 108. The vessel 120 may represent fluid filled or surrounded structures, both natural and man-made. The vessel 120 may be within a body of a patient. The vessel 120 may be a blood vessel, as an artery or a vein of a patient's vascular system, including cardiac vasculature, peripheral vasculature, neural vasculature, renal vasculature, and/or or any other suitable lumen inside the body. For example, the intravascular device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the intravascular device 102 may be used to examine man-made structures such as, but without limitation, heart valves, stents, shunts, filters and other devices.

In some embodiments, the intravascular device 102 includes some features similar to traditional solid-state IVUS catheters, such as the EagleEye® catheter available from Volcano Corporation and those disclosed in U.S. Pat. No. 7,846,101 hereby incorporated by reference in its entirety. For example, the intravascular device 102 includes the physiologic sensor assembly 110 near a distal end of the intravascular device 102 and an electrical cable 112 extending along the longitudinal body of the intravascular device 102. The cable 112 is a transmission line bundle including a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 American wire gauge (AWG) wires. In an embodiment, the cable 112 can include a seven-conductor transmission line arrangement utilizing, e.g., 44 AWG wires. In some embodiments, 43 AWG wires can be used.

The cable 112 terminates in a PIM connector 114 at a proximal end of the intravascular device 102. The PIM connector 114 electrically couples the cable 112 to the PIM 104 and physically couples the intravascular device 102 to the PIM 104. In an embodiment, the intravascular device 102 further includes a guide wire exit port 116 disposed near a junction 130 at which a distal portion 131 is coupled to a proximal portion 132. Accordingly, in some instances the IVUS device is a rapid-exchange catheter. The guide wire exit port 116 allows a guide wire 118 to be inserted towards the distal end in order to direct the intravascular device 102 through the vessel 120.

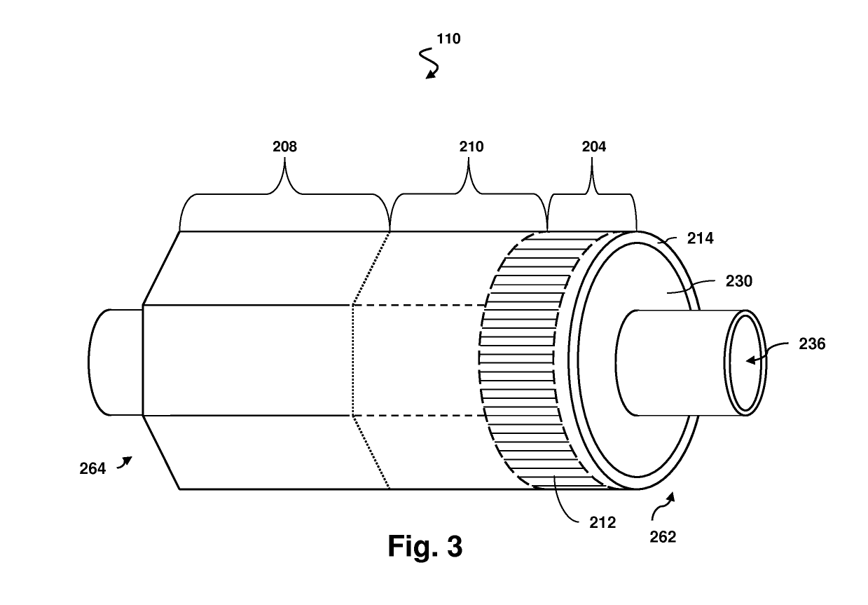

FIG. 2 is a diagrammatic top view of a portion of a physiologic sensor assembly 110, according to aspects of the present disclosure. The physiologic sensor assembly 110 includes a transducer array 124 formed in a transducer region 204 and transducer control logic dies 206 (including dies 206A and 206B) formed in a control region 208, with a transition region 210 disposed therebetween. The transducer array 124 includes an array of IVUS transducers 212. The transducer control logic dies 206 and the transducers 212 are mounted on a flex circuit 214 that is shown in a flat configuration in FIG. 2. Although the physiologic sensor assembly 110 shown in FIG. 2 is an IVUS imaging assembly, it is understood that the physiologic sensor assembly 110 may be configured to obtain any type of physiologic data. FIG. 3 illustrates a rolled configuration of the flex circuit 214. The transducer array 124 is a non-limiting example of a medical sensor element and/or a medical sensor element array. The transducer control logic dies 206 is a non-limiting example of a control circuit. The transducer region 204 is disposed adjacent a distal portion 228 of the flex circuit 214. The control region 208 is disposed adjacent the proximal portion 222 of the flex circuit 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or a length 227 of the transition region 210 can be greater than lengths 225, 229 of the transducer region and controller region, respectively. While the physiologic sensor assembly 110 is described as including a flex circuit, it is understood that the transducers and/or controllers may be arranged to form the physiologic sensor assembly 110 in other configurations, including those omitting a flex circuit.

The transducer array 124 may include any number and type of ultrasound transducers 212, although for clarity only a limited number of ultrasound transducers are illustrated in FIG. 2. In an embodiment, the transducer array 124 includes 64 individual ultrasound transducers 212. In a further embodiment, the transducer array 124 includes 32 ultrasound transducers 212. Other numbers are both contemplated and provided for. With respect to the types of transducers, in an embodiment, the ultrasound transducers 212 are piezoelectric micromachined ultrasound transducers (PMUTs) fabricated on a microelectromechanical system (MEMS) substrate using a polymer piezoelectric material, for example as disclosed in U.S. Pat. No. 6,641,540, which is hereby incorporated by reference in its entirety. In alternate embodiments, the transducer array includes piezoelectric zirconate transducers (PZT) transducers such as bulk PZT transducers, capacitive micromachined ultrasound transducers (cMUTs), single crystal piezoelectric materials, other suitable ultrasound transmitters and receivers, and/or combinations thereof.

The physiologic sensor assembly 110 may include various transducer control logic, which in the illustrated embodiment is divided into discrete control logic dies 206. In various examples, the control logic of the physiologic sensor assembly 110 performs: decoding control signals sent by the PIM 104 across the cable 112, driving one or more transducers 212 to emit an ultrasonic signal, selecting one or more transducers 212 to receive a reflected echo of the ultrasonic signal, amplifying a signal representing the received echo, and/or transmitting the signal to the PIM across the cable 112. In the illustrated embodiment, a physiologic sensor assembly 110 having 64 ultrasound transducers 212 divides the control logic across nine control logic dies 206, of which five are shown in FIG. 2. Designs incorporating other numbers of control logic dies 206 including 8, 9, 16, 17 and more are utilized in other embodiments. In general, the control logic dies 206 are characterized by the number of transducers they are capable of driving, and exemplary control logic dies 206 drive 4, 8, and/or 16 transducers.

The control logic dies are not necessarily homogenous. In some embodiments, a single controller is designated a master control logic die 206A and contains the communication interface for the cable 112. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

The flex circuit 214, on which the transducer control logic dies 206 and the transducers 212 are mounted, provides structural support and interconnects for electrical coupling. The flex circuit 214 may be constructed to include a film layer of a flexible polyimide material such as KAPTON™ (trademark of DuPont). Other suitable materials include polyester films, polyimide films, polyethylene napthalate films, or polyetherimide films, other flexible printed semiconductor substrates as well as products such as Upilex® (registered trademark of Ube Industries) and TEFLON® (registered trademark of E.I. du Pont). In the flat configuration illustrated in FIG. 2, the flex circuit 214 has a generally rectangular shape. As shown and described herein, the flex circuit 214 is configured to be wrapped around a support member 230 (FIG. 3) to form a cylindrical toroid in some instances. Therefore, the thickness of the film layer of the flex circuit 214 is generally related to the degree of curvature in the final assembled physiologic sensor assembly 110. In some embodiments, the film layer is between 5 μm and 100 μm, with some particular embodiments being between 12.7 μm and 25.1 μm.

To electrically interconnect the control logic dies 206 and the transducers 212, in an embodiment, the flex circuit 214 further includes conductive traces 216 formed on the film layer that carry signals between the control logic dies 206 and the transducers 212. In particular, the conductive traces 216 providing communication between the control logic dies 206 and the transducers 212 extend along the flex circuit 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flex circuit 214. Suitable materials for the conductive traces 216 include copper, gold, aluminum, silver, tantalum, nickel, and tin, and may be deposited on the flex circuit 214 by processes such as sputtering, plating, and etching. In an embodiment, the flex circuit 214 includes a chromium adhesion layer. The width and thickness of the conductive traces 216 are selected to provide proper conductivity and resilience when the flex circuit 214 is rolled. In that regard, an exemplary range for the thickness of a conductive trace 216 and/or conductive pad is between 10-50 μm. For example, in an embodiment, 20 μm conductive traces 216 are separated by 20 μm of space. The width of a conductive trace 216 on the flex circuit 214 may be further determined by the width of the conductor 218 to be coupled to the trace/pad.

The flex circuit 214 can include a conductor interface 220 in some embodiments. The conductor interface 220 can be a location of the flex circuit 214 where the conductors 218 of the cable 112 are coupled to the flex circuit 214. For example, the bare conductors of the cable 112 are electrically coupled to the flex circuit 214 at the conductor interface 220. The conductor interface 220 can be tab extending from the main body of flex circuit 214. In that regard, the main body of the flex circuit 214 can refer collectively to the transducer region 204, controller region 208, and the transition region 210. In the illustrated embodiment, the conductor interface 220 extends from the proximal portion 222 of the flex circuit 214. In other embodiments, the conductor interface 220 is positioned at other parts of the flex circuit 214, such as the distal portion 228, or the flex circuit 214 omits the conductor interface 220. A value of a dimension of the tab or conductor interface 220, such as a width 224, can be less than the value of a dimension of the main body of the flex circuit 214, such as a width 226. In some embodiments, the substrate forming the conductor interface 220 is made of the same material(s) and/or is similarly flexible as the flex circuit 214. In other embodiments, the conductor interface 220 is made of different materials and/or is comparatively more rigid than the flex circuit 214. For example, the conductor interface 220 can be made of a plastic, thermoplastic, polymer, hard polymer, etc., including polyoxymethylene (e.g., DELRIN®), polyether ether ketone (PEEK), nylon, and/or other suitable materials. As described in greater detail herein, the support member 230, the flex circuit 214, the conductor interface 220 and/or the conductor(s) 218 can be variously configured to facilitate efficient manufacturing and operation of the physiologic sensor assembly 110.

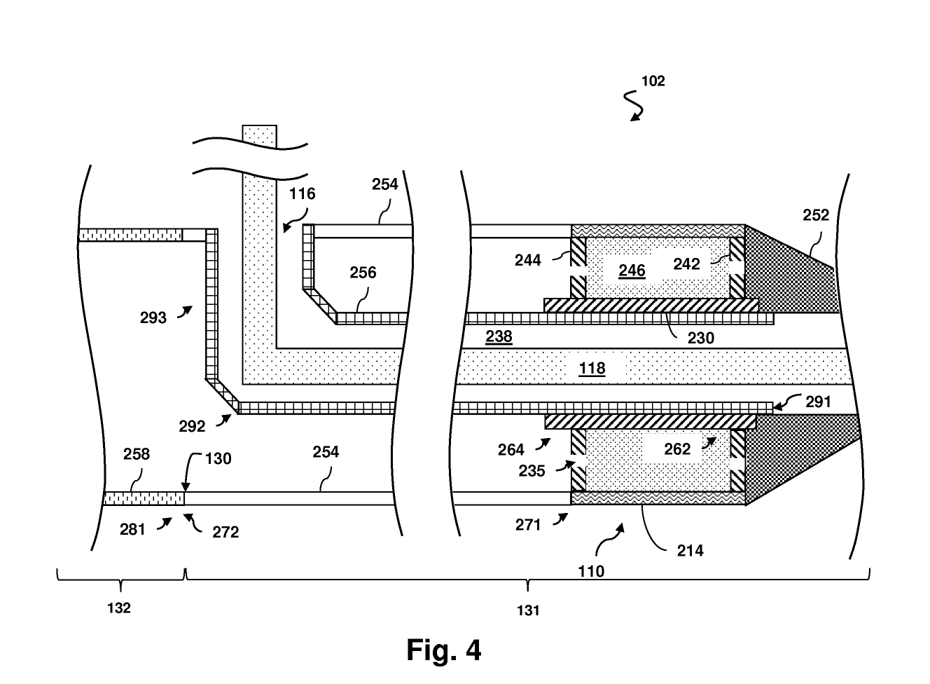

In some instances, the physiologic sensor assembly 110 is transitioned from a flat configuration (FIG. 2) to a rolled or more cylindrical configuration (FIGS. 3 and 4). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

As shown in FIGS. 3 and 4, the flex circuit 214 is positioned around the support member 230 in the rolled configuration. FIG. 3 is a diagrammatic side view with the flex circuit 214 in the rolled configuration around the support member 230, according to aspects of the present disclosure. FIG. 4 is a diagrammatic cross-sectional side view of the intravascular device 102, according to aspects of the present disclosure. While FIGS. 3 and 4 may illustrate the intravascular device 102 in the context of an IVUS device, the intravascular device 102 and/or the physiologic sensor assembly 110 can obtain various different types of intravascular data.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, ('220 Application) the entirety of which is hereby incorporated by reference herein. The support member 230 can be a ferrule having a distal portion 262 and a proximal portion 264. The support member 230 can be a ferrule having a distal portion 262 and a proximal portion 264. The support member 230 can be tubular in shape and define a lumen 236 extending longitudinally therethrough. The lumen 236 is sized and shaped to receive a distal inner member 256 (FIG. 4). The support member 230 can be manufactured accordingly to any suitable process. For example, the support member 230 can be machined, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another.

In some embodiments, stands 242, 244 extending vertically are provided at the distal and proximal portions 262, 264, respectively, of the support member 230. For example, the stands 242, 244 elevate and support the distal and proximal portions of the flex circuit 214. In that regard, portions of the flex circuit 214, such as the transducer region 204, can be spaced from a central body portion of the support member 230 extending between the stands 242, 244. To improve acoustic performance, any cavities between the flex circuit 214 and the surface of the support member 230 are filled with a backing material 246. The liquid backing material 246 can be introduced between the flex circuit 214 and the support member 230 via passageways 235 in the stands 242, 244.

As shown in FIG. 4, the intravascular device 102 includes a distal portion 131 and a proximal portion 132. The distal portion 131 can be referred to as a distal shaft. The proximal portion 132 can be referred to as a proximal shaft. Together, the distal shaft and the proximal shaft may form a flexible elongate member of the intravascular device 102. In some instances, the proximal portion 132 can include a mid shaft and a proximal shaft. The distal shaft can include a distal outer member 254 and a distal inner member 256. The proximal shaft can include a proximal outer member 258. For example, the distal outer member 254, the distal inner member 256, and the proximal outer member 258 can be flexible elongate members and may be tubular in shape. The distal outer member 254 includes a distal end 271 and a proximal end 272. The proximal outer member 258 includes a distal end 281 and a proximal end (not shown), which can be connected to the PIM connector 114. The proximal end 272 of the distal outer member 254 abuts and is in contact with the distal end 281 of the proximal outer member 258 at the coupling junction 130. The distal end 271 of the distal outer member 254 is coupled to the physiologic sensor assembly 110. For example, the distal end 271 can abut and be in contact with the flex circuit 214.

The distal inner member 256 includes a distal end 291 and a proximal end 292. The distal inner member 256 extends longitudinally through the distal outer member 254 and is coupled to the physiologic sensor assembly 110. For example, the distal inner member 256 can be coupled to the support member 230. For example, the distal inner member 256 can extend longitudinally through and distally beyond the lumen 236 of the support member 230 such that the distal end 291 of the distal inner member 256 protrudes from the support member 230. The distal inner member 256 can define a lumen 238 extending longitudinally therethrough. The distal inner member 252 can couple to the support member 230 and/or a distal tip member 252. In some embodiments, the distal inner member 256 can include a portion 293 that extends towards the exit port 116 so that the lumen 238 of the distal inner member 256 is in communication with the exit port 116. In such embodiments, the lumen 238 of the distal inner member 256 can be sized and shaped to receive the guide wire 118 (FIG. 1).

The distal outer member 254, the distal inner member 256, and the proximal outer member 258 can be composed of a material such as plastic, polymer, metal, other suitable materials, and/or combinations thereof. As described herein, the dimensions of the distal outer member 254, the distal inner member 256, and the proximal outer member 258 can vary in different embodiments.

The distal tip member 252 is coupled to a distal end 291 of the distal inner member 256 and the distal portion 262 of the support member 230. The distal tip member 252 can be a flexible component that defines a distal most portion of the intravascular device 102. The distal tip member 252 can couple to the flex circuit 214, the stands 242, the support member 230, and/or the distal inner member 256. The distal tip member 252 can abut and be in contact with the flex circuit 214 and the stand 242. The distal tip member 252 can be the distal-most component of the intravascular device 102.

One or more adhesives can be disposed between various components of the intravascular device 102. For example, one or more of the flex circuit 214, the support member 230, the distal tip member 252, the distal inner member 256, the distal outer member 254, and/or the proximal outer member 258 can be coupled to one another via an adhesive.

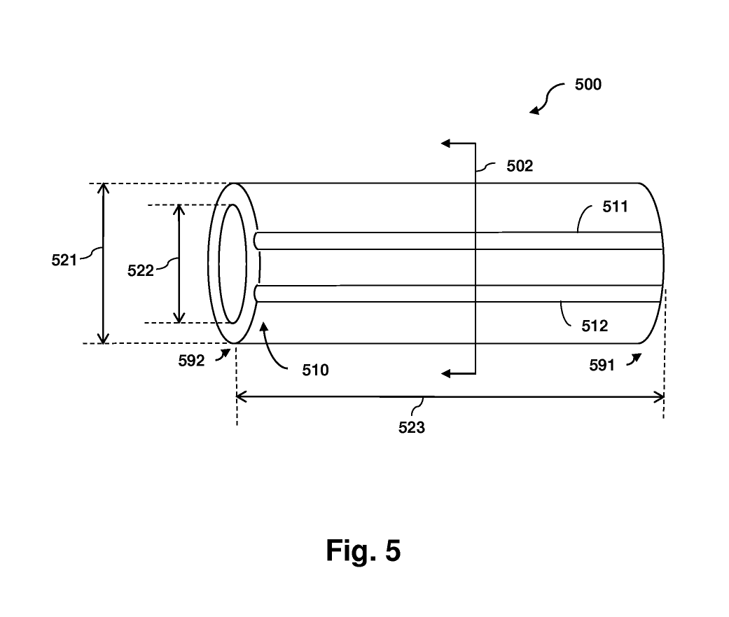

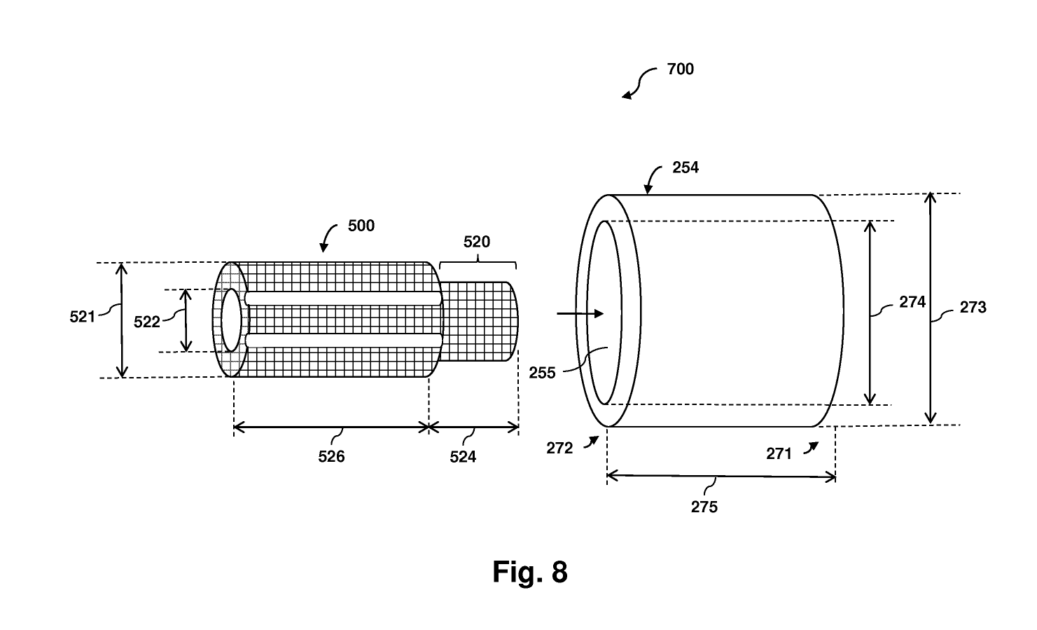

FIG. 5 is a diagrammatic perspective view of a distal inner member 500, according to aspects of the present disclosure. The distal inner member 500 can be similar in some respects to the distal inner member 256 and can be used by the intravascular device 102 in place of the distal inner member 256 within the distal outer member 254. The distal inner member 500 can be composed of similar materials as the distal inner member 256. The distal inner member 500 can be tubular in shape and include a distal end 591 and a proximal end 592. In some embodiments, the distal inner member 500 can have an outer diameter 521 between about 0.03 inch and about 0.033 inch when the distal outer member 254 has an inner diameter between about 0.032 inch and 0.035 inch, and/or other suitable values. The distal inner member 500 can have an inner diameter 522 of about 0.017 inch and a length 523 of about 30 cm, and/or other suitable values. The length 523 of the distal inner member 500 may be greater than a length 275 of the distal outer member 254 (FIG. 8) in some instances.

Groves 511 and 512 are formed on an outer surface 510 of the distal inner member 500. The groves 511 and 512 are also referred to as channels. The grooves 511 and 512 extend along a longitudinal length of the distal inner member 500. Although the grooves 511 and 512 are illustrated to extend an entire length from the proximal end 592 to the distal end 591, the grooves 511 or 512 can extend any suitable length along the distal inner member 500. The groves 511 and 512 are circumferentially spaced from one another and can be separated by any suitable distance on the outer surface 510. The groves 511 and 512 can have the same dimension or different dimensions. The grooves 511 and 512 can be shaped and sized to accommodate installation of cables and/or wires within the grooves 511 and 512. For example, the electrical cable 112 can be aligned and positioned within the grove 511 and a stiffening wire can be aligned and positioned within the groove 512, as described more fully below. The length of the groove 511 or 512 can be based on the length of the stiffening wire. For example, the groove 511 or 512 can terminate at the end of the stiffening wire. In some embodiments, the distal inner member 500 can include two, three, four, five, or any suitable number of grooves similar to the grooves 511 and 512. The grooves 511 and 512 can extend radially into the distal inner member 500 from the outer surface 510 by any suitable amount. For example, a dimension 515 of the groove 512 can be about 0.012 inch and/or other suitable values. A width 517 of the groove 512 can be between about 0.5 millimeter (mm) and about 0.75 mm, and/or other suitable values. While the grooves 511 and 512 are illustrated has having a generally oval or elliptical shape, it is understood the grooves can have any suitable shape including a circular shape, a polygonal shape, etc.

The distal inner member 500 can be manufactured accordingly to any suitable process. For example, the distal inner member 500 can be machined, such as by removing material from a blank to shape the distal inner member 500, or molded, such as by an injection molding process. In some embodiments, the distal inner member 500 may be integrally formed with the grooves 511 and 512 as a unitary structure, while in other embodiments the distal inner member 500 may be formed by removing material from the outer surface 510 to form the grooves 511 and 512. In some embodiments, the distal inner member 500 can be extended to couple to the guide wire exit port 116 as shown in FIG. 4.

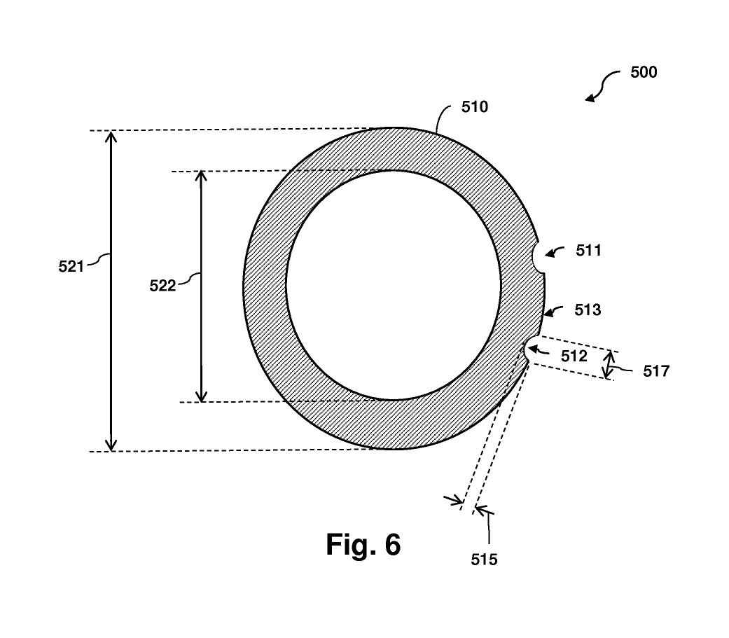

FIG. 6 is a diagrammatic schematic cross-sectional view of the distal inner member 500 taken along the line 502 of FIG. 5, according to aspects of the present disclosure. As shown, the groves 511 and 512 are recessed from the outer surface 510 or sidewall of the distal inner member 500 and separated by a rib 513.

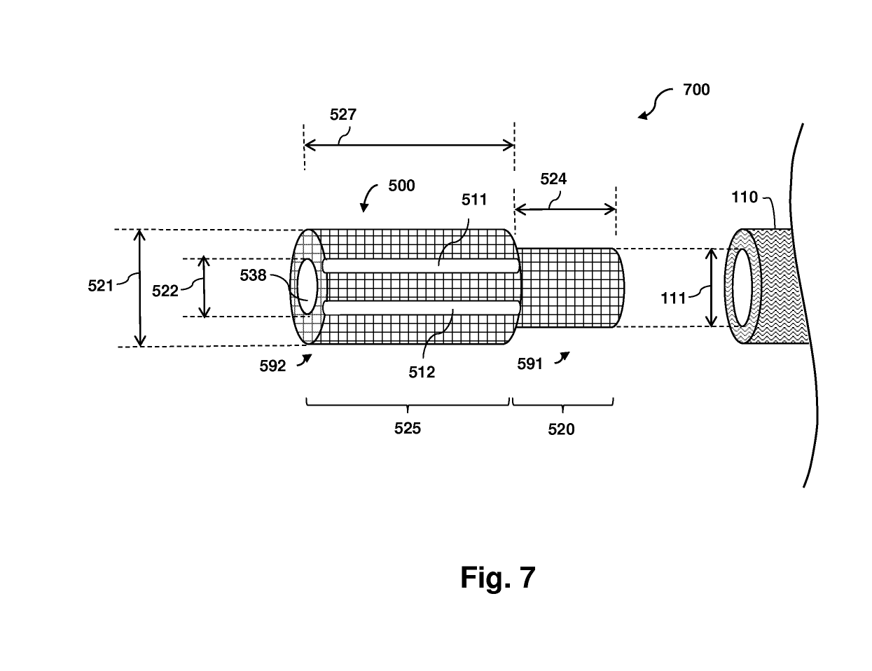

FIGS. 7-13 collectively illustrate a method 700 of assembling the intravascular device 102 including the distal inner member 500. The method 700 employs similar mechanisms as methods 1500 and 1600. FIG. 7 is a diagrammatic perspective view of the distal inner member 500 sized and shaped to fit into the inner diameter of the physiologic sensor assembly 110, according to aspects of the present disclosure. For example, the distal inner member 500 can have an outer diameter 521 greater than an inner diameter 111 of the physiologic sensor assembly 110. In some embodiments, the inner diameter 111 of the physiologic sensor assembly 110 may be about 0.55 mm, and/or other suitable values. For example, the method 700 includes applying heat to the distal inner member 500 at the distal end 591 and passing the distal end 591 through a die and/or otherwise molding the distal end 591 to reduce an outer diameter 521 of the distal inner member 500 at the distal end 591. In some embodiments, the method 700 can include inserting a mandrel into the lumen 538 of the distal inner member 500 prior to the heating. The positioning of the mandrel in the lumen 538 preserves the inner diameter 522 of the lumen 538 along an entire length of the distal inner member 500 during the heating. Thus, the heating reduces the thickness of the distal inner member 500 sidewall while maintaining the inner diameter 522 of the lumen 538. After reducing the thickness of the sidewall of the distal inner member 500, the mandrel is removed. As shown, a section 520 of the distal inner member 500 at the distal end 591 has a smaller outer diameter and a thinner sidewall than the remaining portion 525 of the distal inner member 500. For example, the outer diameter of the section 520 is substantially similar to the inner diameter 111 of the physiologic sensor assembly 110. In some embodiments, the reduced section 520 may have a length 524 between about 2 centimeter (cm) and about 3 cm, and/or suitable values. In some embodiments, the remaining portion 525 may have a length 527 between about 25 cm and about 30 cm. In some instances, the reduced section 520 includes grooves 511 and 512. In other instances, heating and passing the distal end 591 through a die eliminates the grooves 511 and 512 in the section 520, while the grooves 511 and 512 remain in portion 525.

FIG. 8 is a diagrammatic perspective view of the distal inner member 500 positioned for moving into the distal outer member 254, according to aspects of the present disclosure. After reducing the outer diameter 521 to create the section 520 at the distal inner member 500 as shown in FIG. 7, the method 700 includes translating or sliding the distal inner member 500 into a lumen 255 of the distal outer member 254. For example, the section 520 enters the lumen 255 from the proximal end 272 of the distal outer member 254 and continues until the section 520 extends beyond the distal end 271 of the distal outer member 254. In some embodiments, the distal outer member 254 may have an outer diameter 273 between about 0.091 cm and about 0.109 cm and an inner diameter 274 between about 0.032 mm and about 0.035 mm when a diameter of an imaging scanner in the physiologic sensor assembly 110 is between about 0.04 inch and about 0.0455 inch, and/or other suitable values. The distal outer member 254 may have an outer diameter 273 between about 0.091 cm and about 0.109 cm, and a length 275 between about 25 cm and about 30 cm. The length 275 of the distal outer member 254 can be substantially similar to the length 526 of the distal inner member 256.

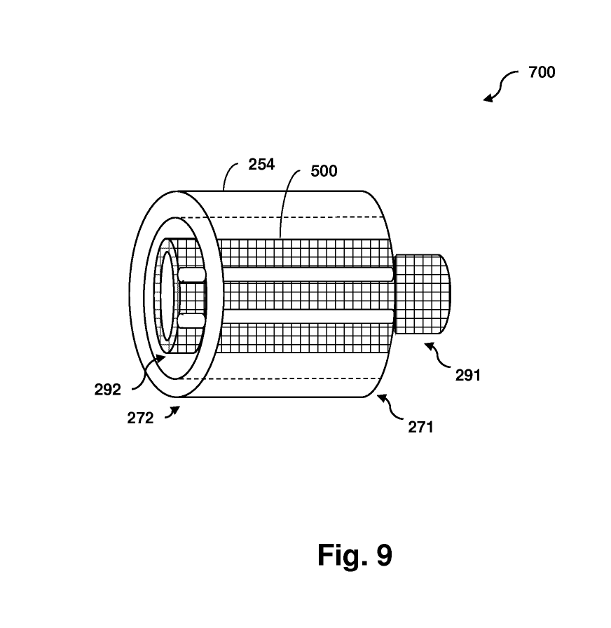

FIG. 9 is a diagrammatic perspective view of the distal inner member 500 positioned in the distal outer member 254 after completing the moving shown in FIG. 8, according to aspects of the present disclosure. As shown, the section 520 of the distal inner member 500 extends distally beyond the distal end 271 of the distal outer member 254 and the proximal end 292 of the distal inner member 500 terminates near the proximal end 272 of the distal outer member 254.

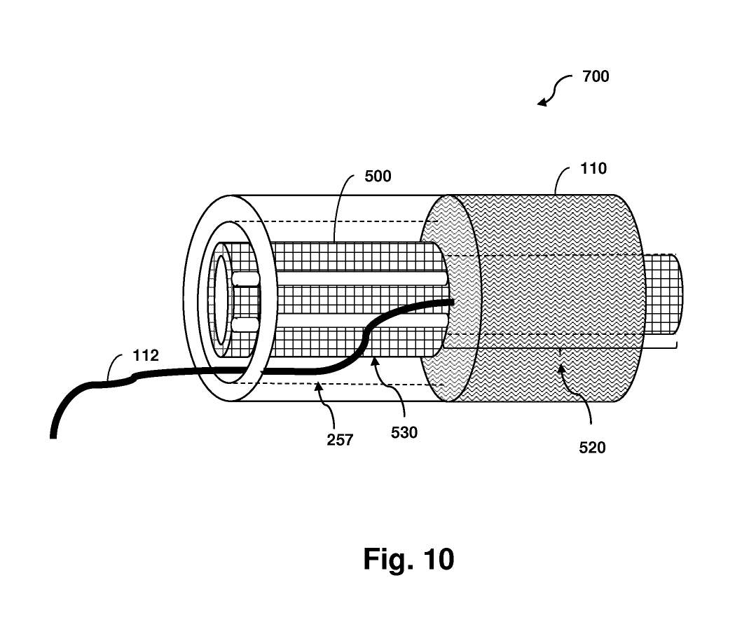

FIG. 10 is a diagrammatic perspective view of the distal inner member 500 bonded to the physiologic sensor assembly 110, according to aspects of the present disclosure. After positioning the distal inner member 500 in the distal outer member 254 as shown in FIG. 9, the 700 includes moving, such as by sliding, rotating, orienting, or translating, the section 520 of the distal inner member 500 into the physiologic sensor assembly 110 such that the section 520 extends distally beyond the physiologic sensor assembly 110. In addition, the method 700 includes bonding the distal inner member 500 to the physiologic sensor assembly 110, for example, using an adhesive. The physiologic sensor assembly 110 can be pre-assembled with the electrical cable 112 as shown in FIG. 2. In some instances, the electrical cable 112 is aligned with the groove 511 or 512 prior to the distal inner member 256 and the distal outer member 254 are bonded to the physiologic sensor assembly 110. For example, the distal inner member 256, the distal outer member 254, the physiologic sensor assembly 110, and/or the electrical cable 112 may be moved relative to one another to facilitate alignment of the electrical cable 112 within the groove 511 or 512. After the bonding, the electrical cable 112 can be floating in a space between the inner sidewall 257 of the distal outer member 254 and the outer sidewall 530 of the distal inner member 500.

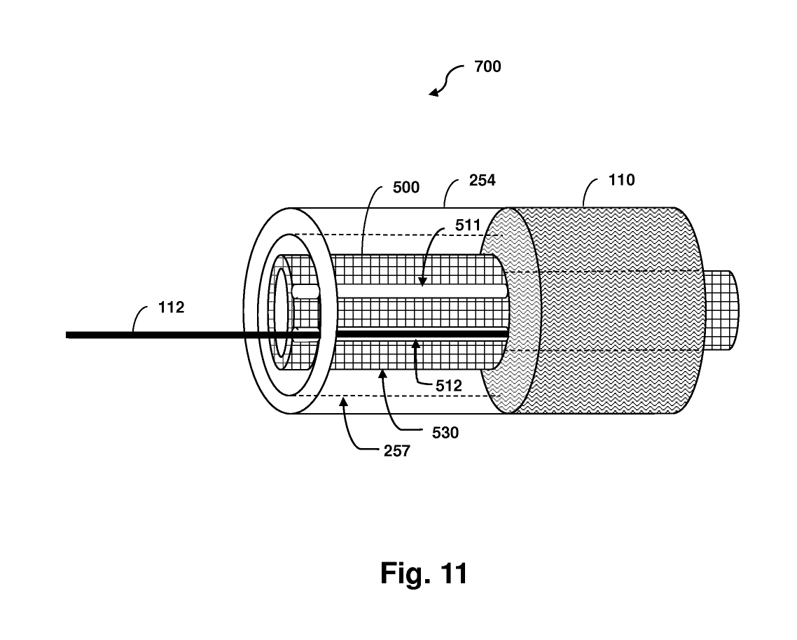

FIG. 11 is a diagrammatic perspective view of the electrical cable 112 of the physiologic sensor assembly 110 positioned within the groove 512 of the distal inner member 500, according to aspects of the present disclosure. After bonding the physiologic sensor assembly 110 to the distal inner member 500 as shown in FIG. 10, the method 700 includes aligning the electrical cable 112 into the groove 512. In some instances, the distal inner member 500 and/or the distal outer member 254 can be rotated to reorient the groove 512 into alignment with the cable 112. In some instances, the sensor assembly 110 is rotated to reorient the cable 112 into alignment with the groove 512. After the cable 112 is aligned with the groove 512, the cable 112 can be positioned within the groove 512. For example, the cable 112 can be press fit into the groove 512 and/or bonded to the groove 512 using an adhesive. In some embodiments, the electrical cable 112 can be positioned within the groove 511 instead of the groove 512. By positioning the electrical cable 112 within the grove 512, the space between the inner sidewall 257 of the distal outer member 254 and the outer sidewall 530 of the distal inner member 500 can be reduced, thus allowing for a smaller-sized or lower-profiled intravascular device 102.

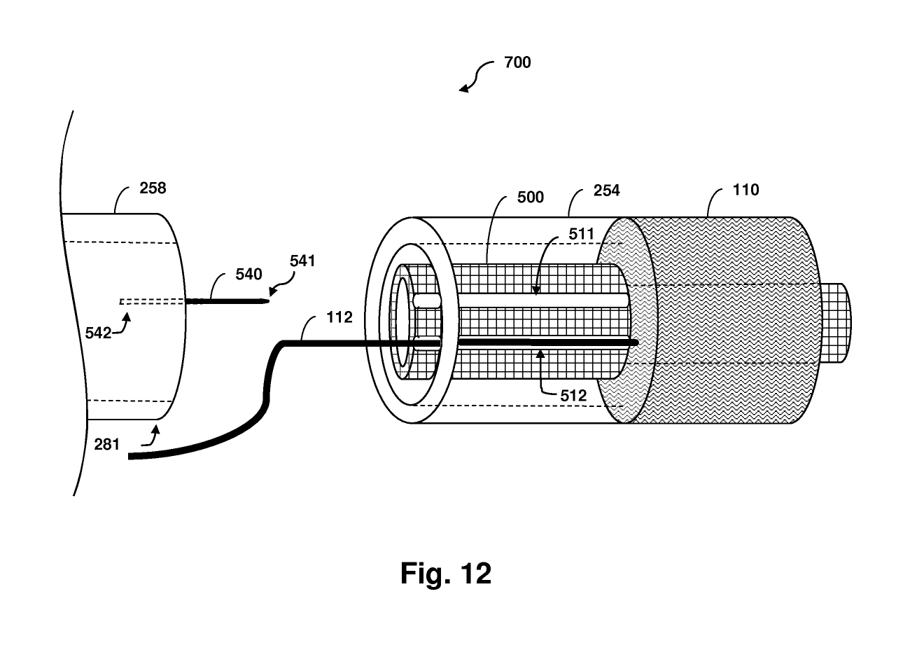

FIG. 12 is a diagrammatic perspective view of a stiffening wire 540 positioned for alignment into the groove 511 of the distal inner member 500, according to aspects of the present disclosure. The stiffening wire 540 includes a proximal end 542 and a distal end 541. The stiffening wire 540 can include a tapered portion near the distal end 541. For example, the stiffening wire 540 can have a thickness of between about 0.02 mm and about 0.15 mm at the proximal end 542, which may be tapered to a thickness between about 0.015 mm and about 0.2 mm at the distal end 541. The stiffening wire 540 can be pre-assembled with the proximal outer member 258. For example, the proximal end 542 of the stiffening wire 540 can be attached to an inner sidewall or inner surface of the proximal outer member 258 near the distal end 281 of the proximal outer member 258 as shown. Alternatively, the stiffening wire 540 can extend through an entire length of the proximal outer member 258. As described above, in some embodiments, the electrical cable 112 can be positioned in the groove 511 instead of the groove 512. In such embodiments, the stiffening wire 540 can be aligned to the groove 512 instead of the groove 511.

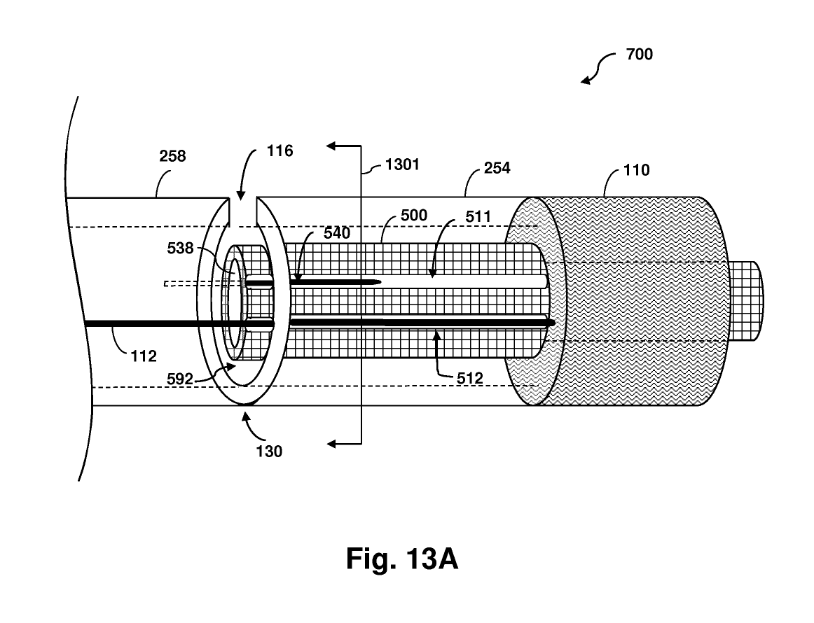

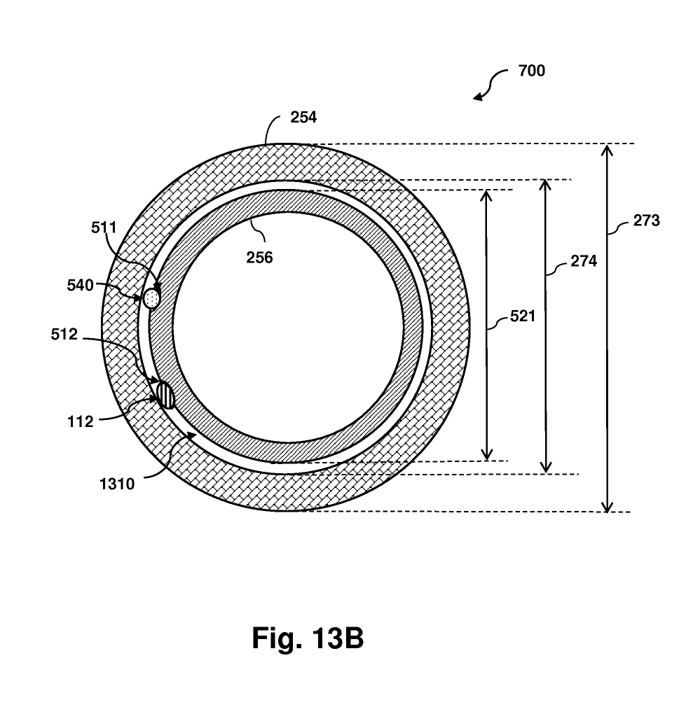

FIG. 13A is a diagrammatic perspective view of the proximal outer member 258 assembled with the distal outer member 254, according to aspects of the present disclosure. After positioning and fitting the stiffening wire 540 within the groove 511, the method 700 includes coupling the proximal outer member 258 to the distal outer member 254, for example, using an adhesive. In addition, during the coupling, the method 700 includes creating the guide wire exit port 116 at the coupling junction 130 between the proximal outer member 258 and the distal outer member 254. In some embodiments, a portion of the distal inner member 500 near the proximal end 592 can be positioned such that the lumen 538 of the distal inner member 500 is in communication with the guide wire exit port 116 as shown in FIG. 4 to enable receiving of the guide wire 118.

As shown in FIG. 13A, the stiffening wire 540 and the electrical cable 112 are positioned within the grooves 511 and 512, respectively, of the distal inner member 500. By including the stiffening wire 540 near the distal end 281 of the proximal outer member 258 and extending the stiffening wire 540 into at least a portion of the distal outer member 254, for example, by about 3 cm, the torque performance of the intravascular device 102 can be improved. Torque performance refers to the rotational, directional, guiding, and/or navigational capabilities of the intravascular device 102. For example, an intravascular device with a high torque performance allows a physician or a user to easily and precisely guide and maneuver the intravascular device through various vascular regions of a vessel within a patient body. Although the stiffening wire 540 is shown to terminate before reaching the distal end 271 of the distal outer member 254, the stiffening wire 540 can terminate at any distance from the distal end 271. The length of the stiffening wire 540 can be dependent on the thickness or the diameter of the stiffening wire 540. For example, a stiffening wire with a greater diameter can have a shorter length than a stiffening wire with a smaller diameter so that the distal outer member 254 can maintain sufficient flexibility for maneuvering during a catheterization procedure.

FIG. 13B is a diagrammatic cross-sectional view of the distal outer member 254, the distal inner member 256, the electrical cable 112, and the stiffening wire 540 coupled in position taken along the line 1301 of FIG. 13, according to aspects of the present disclosure. As shown, the electrical cable 112 and the stiffening wire 540 are fitted within the grooves 511 and 512, respectively. As such, the space 1310 between the distal outer member 254 and the distal inner member 256 can be efficiently utilized. Thus, the inner diameter 274 of the distal outer member 254 can be reduced. For example, the inner diameter 274 of the distal outer member 254 can be between about 0.081 mm and between about 0.089 mm greater than the outer diameter 521 of the distal inner member 256 when a diameter of an imaging scanner in the physiologic sensor assembly 110 is between about 0.04 inch and about 0.0455 inch. Thus, the outer diameter 273 and the overall size or outer profile of the intravascular device 102 can also be reduced. For example, the disclosed embodiments can provide a lower profile solid-state intravascular device having a diameter in a range between about 3 French (Fr) to about 3.5 Fr. Conventional solid-state intravascular devices, on the other hand, have a diameter in a range between about 3 Fr to about 3.5 Fr.

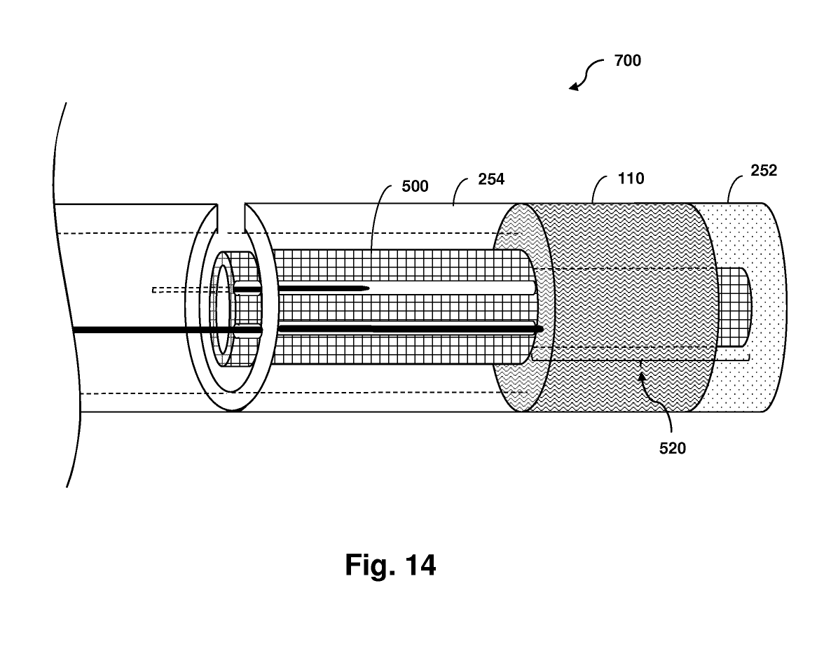

FIG. 14 is a diagrammatic perspective view of the distal tip member 252 bonded to the distal inner member 500, according to aspects of the present disclosure. After coupling the proximal outer member 258 to the distal outer member 254, the method 700 includes coupling and bonding the distal tip member 252 to the section 520 of the distal inner member 500, for example, using an adhesive. The distal tip member 252 can also be coupled to the sensor assembly 110 and/or the support member 230 of the sensor assembly 110 (FIG. 4). For example, the distal tip member 252 can surround the protruding portion of the section 520 of the distal inner member 500. The extension and coupling of the distal inner member 500 to the distal tip member 252 can improve rotational or torque quality of intravascular device 102.

As described above, the disclosed embodiments provide several benefits. For example, wires and/or cables typically extend along the length of an intravascular device between an outer member and an inner member. By fitting cables and/or wires such as the electrical cable 112 and the stiffening wire 540 into the grooves 511 and 512 of the distal inner member 256, the space 1310 between the distal outer member 254 and the distal inner member 256 can be efficiently utilized as shown in FIGS. 13A and B, and thus allow for a reduced profile intravascular device. In addition, by inserting the stiffening wire 540 near the junction 130 and extending the distal inner member 256 to the distal tip member 252, the rotational, guiding, and/or directional capabilities of the intravascular device are improved. Further, reducing the outer profile or diameter/size of intravascular imaging devices also allows the device to more efficiently traverse vasculature

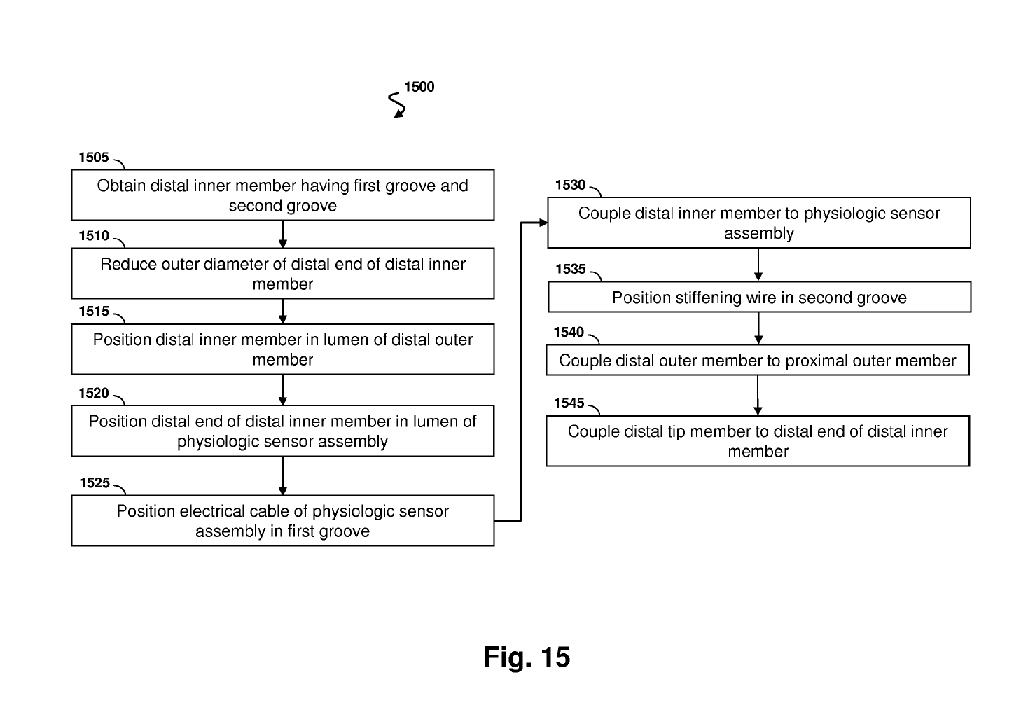

FIG. 15 is a flow diagram of a method 1500 of assembling an IVUS device, including a distal inner member described herein, according to aspects of the present disclosure. It is understood that the steps of the method 1500 may be performed in a different order than shown in FIG. 15, additional steps can be provided before, during, and after the steps, and/or some of the steps described can be replaced or eliminated in other embodiments. The steps of the method 1500 can be carried out by a manufacturer of the IVUS device. The method 1500 can employ similar mechanisms as the methods 700 and 1600. The method 1500 can correspond to the steps described with respect to FIGS. 7-14.

At step 1505, the method 1500 includes obtaining a distal inner member including a first groove and a second groove. The distal inner member can be similar to the distal inner member 500. The first and second grooves can be similar to the grooves 511 and 512. For example, the distal inner member is tubular in shape and includes a distal end and a proximal end, and the first and second grooves extend longitudinally along an outer surface of the distal inner member.

At step 1510, the method 1500 includes reducing an outer diameter of the distal end of the distal inner member. For example, the method 1500 includes positioning a mandrel within a lumen of the distal inner member and applying heat to reduce the outer diameter based on an inner diameter of a lumen of a physiologic sensor assembly. The physiologic sensor assembly can be similar to the physiologic sensor assemble 110. The mandrel can preserve the inner diameter along an entire length of the distal inner member during the heating. After completing the heating, the method 1500 includes removing the mandrel from the distal inner member. After the heating, the distal inner member includes a first inner member portion at the distal end with a smaller outer diameter than a second inner member portion adjacent to the first inner member portion as shown in FIG. 7.

At step 1515, the method 1500 includes positioning the distal inner member in a lumen of a distal outer member. The distal outer member can be similar to the distal outer member 254 and can be sized and shaped for insertion into a vessel of a patient. The distal outer member includes a distal end and a proximal end. For example, the method 1500 includes positioning the distal inner member along a longitudinal axis of the distal outer member such that the first inner member portion extends beyond the distal end of the distal outer member as shown in FIG. 9.

At step 1520, the method 1500 includes positioning the distal end of the distal inner member in the lumen of the physiologic sensor assembly. For example, the method 1500 includes positioning the distal inner member within the physiologic sensor assembly such that the first inner member portion extends through the lumen of the physiologic sensor assembly and distally beyond the physiologic sensor assembly as shown in FIG. 10.

At step 1525, the method 1500 includes positioning an electrical cable of the physiologic sensor assembly in the second groove as shown in FIG. 11. The electrical cable can be similar to the electric cable 112 and can be pre-assembled with the physiologic sensor assembly. The positioning can include rotating and reorienting the electrical cable 112 to facilitate alignment of the electrical cable 112 into the second groove. At step 1530, the method 1500 includes coupling the distal inner member to the physiologic sensor assembly, for example, using an adhesive.

At step 1535, the method 1500 includes positioning a stiffening wire in the first groove. The stiffening wire can be similar to the stiffening wire 540 and can be coupled to a proximal outer member. The proximal outer member can be similar to the proximal outer member 258 and includes a proximal end and a distal end. For example, the stiffening wire 540 is attached to an inner sidewall of the proximal outer member and positioned within the first groove as shown in FIG. 13.

At step 1540, the method 1500 includes coupling the proximal outer member to the proximal end of the distal outer member, for example, using an adhesive. In addition, the method 1500 can include creating a guide wire exit port at a coupling junction between the distal outer member and the proximal outer member during the coupling such that a third inner member portion of the distal inner member adjacent to the second inner member portion is in communication with the guide wire exit port as shown in FIG. 4

At step 1545, the method 1500 includes coupling a distal tip member to the distal end of the distal inner member, for example, using an adhesive. The distal tip member can be similar to the distal tip member 252. For example, the distal tip member can surround the protruding portion of the first inner member portion as shown in FIG. 14.

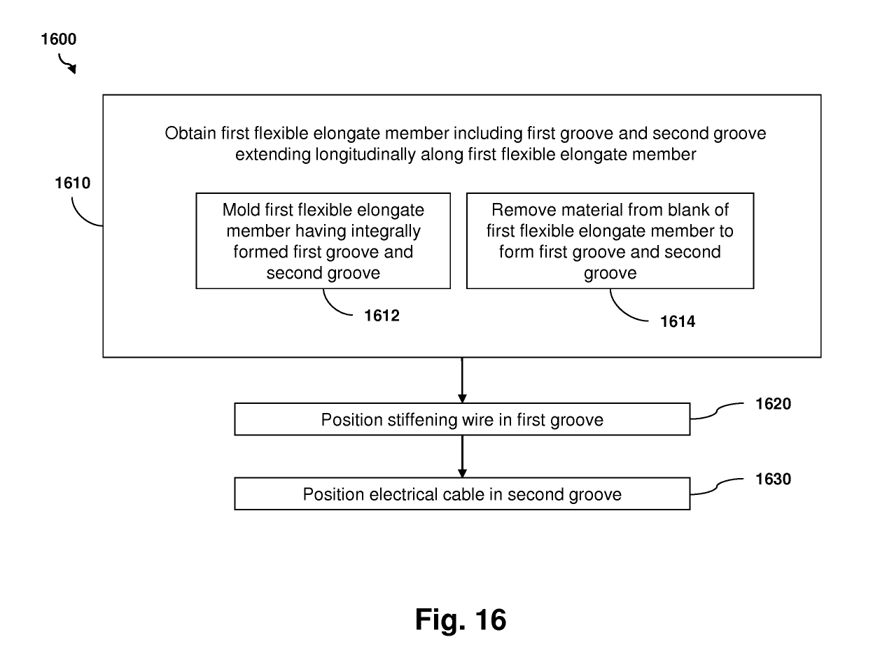

FIG. 16 is a flow diagram of a method 1600 of assembling an IVUS device, including a distal inner member described herein, according to aspects of the present disclosure. It is understood that the steps of the method 1600 may be performed in a different order than shown in FIG. 16, additional steps can be provided before, during, and after the steps, and/or some of the steps described can be replaced or eliminated in other embodiments. The steps of the method 1600 can be carried out by a manufacturer of the IVUS device. The method 1600 can employ similar mechanisms as the methods 700 and 1500. The method 1600 can employ similar mechanisms as the methods 700 and 1600. The method 1600 can correspond to the steps described with respect to FIGS. 7-14.

At step 1610, the method 1600 includes obtaining a first flexible elongate member including a first groove and a second groove extending longitudinally along the first flexible elongate member. The first flexible elongate member can be similar to the distal inner member 500. The first and second grooves are similar to the grooves 511 and 512. In some embodiments, obtaining the first flexible elongate member can include molding the first flexible elongate member (step 1612), such as with injection molding and/or other suitable manufacturing process that integrally forms the first groove and the second groove on the outer surface of the first flexible elongate member. In some embodiments, obtaining the first flexible elongate member can include machining the first flexible elongate member from a blank. For example, step 1610 can include removing material from a blank of the first flexible elongate member to form the first groove and the second groove (step 1614).

At step 1620, the method 1600 includes positioning a stiffening wire in the first groove. The stiffening wire is coupled to a second flexible elongate member. The stiffening wire can be similar to the stiffening wire 540. The second flexible elongate member can be similar to the proximal outer member 258. For example, the stiffening wire is attached or secured to an inner sidewall of the second flexible elongate member as shown in FIG. 12.

At step 1630, the method 1600 includes positioning an electrical cable in the second groove, where the electrical cable is coupled to a physiologic sensor assembly. The electrical cable can be similar to the electrical cable 112. The physiologic sensor assembly can be similar to the physiologic sensor assembly 110. The positioning of the stiffening wire in the first groove and the positioning of the electrical cable in the second groove can be as shown in FIG. 13. The method 1600 can include additional steps as described in the methods 700 and 1500. For example, the method 1600 can also include reducing an outer diameter of a distal end of the first flexible elongate member. The method 1600 can include positioning the first flexible elongate member in a lumen of a third flexible elongate member similar to distal outer member 254. The method 1600 can include positioning the distal end of the first flexible elongate member in a lumen of the physiologic sensor assembly. The method 1600 can include coupling and bonding the first flexible elongate member to the physiologic sensor assembly. The method 1600 can include coupling and/or bonding the third flexible elongate member to the second flexible elongate member. The method 1600 can include coupling a distal tip member similar to the distal tip member 252 to the distal end of the first flexible elongate member.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intravascular device, comprising:
a flexible elongate member sized and shaped for insertion into a vessel of a patient, the flexible elongate member having a distal portion and a proximal portion; and
a physiologic sensor assembly disposed at the distal portion,
wherein the distal portion of the flexible elongate member comprises a distal inner member coupled to the physiologic sensor assembly, the distal inner member including a first groove extending longitudinally along the distal inner member, and
wherein one of a stiffening wire extending longitudinally within the flexible elongate member or an electrical cable coupled to the physiologic sensor assembly is positioned within the first groove of the distal inner member.

2. The intravascular device of claim 1, wherein the distal inner member is tubular in shape, and wherein the first groove extends longitudinally along an outer surface of the distal inner member.

3. The intravascular device of claim 2, wherein the distal inner member includes a second groove extending longitudinally along the outer surface, and wherein the other of the stiffening wire or the electrical cable is positioned within the second groove of the distal inner member.

4. The intravascular device of claim 3, wherein the first groove and the second groove are circumferentially spaced from one another.

5. The intravascular device of claim 1, wherein the distal inner member includes:
a first distal end;
a first proximal end;
a first inner member portion at the first distal end and coupled to the physiologic sensor assembly; and
a second inner member portion adjacent to the first inner member portion.

6. The intravascular device of claim 5, wherein the first groove extends longitudinally along a length of an outer surface of the second inner member portion.

7. The intravascular device of claim 5, wherein the flexible elongate member comprises:
a proximal outer member disposed at the proximal portion of the flexible elongate member; and
a distal outer member disposed at the distal portion of the flexible elongate member, wherein the distal outer member includes a second proximal end coupled to the proximal outer member and a second distal end coupled to the physiologic sensor assembly,
wherein the distal inner member extends longitudinally through the distal outer member, and
wherein the stiffening wire is coupled to the proximal outer member.

8. The intravascular device of claim 7, wherein the proximal outer member is tubular in shape, and wherein the stiffening wire includes:
a third proximal end attached to an inner surface of the proximal outer member; and
a third distal end positioned within the first groove of the distal inner member.

9. The intravascular device of claim 7, wherein the distal inner member includes a third inner member portion adjacent to the second inner member portion, and wherein the third inner member portion defines a guide wire exit port disposed at a coupling junction between the proximal outer member and the distal outer member.

10. The intravascular device of claim 5, wherein the physiologic sensor assembly comprises an array of intravascular ultrasound (IVUS) transducers disposed on a flex circuit positioned circumferentially around a support member, and wherein the first inner member portion extends through a lumen of the support member and distally beyond the support member.

11. The intravascular device of claim 5, wherein the distal inner member is tubular in shape, and wherein the second inner member portion has a greater outer diameter than the first inner member portion.

12. The intravascular device of claim 5, further comprising a distal tip member coupled to the first distal end of the distal inner member.

13. A method of assembling an intravascular device, the method comprising:
obtaining a first flexible elongate member including a first groove and a second groove extending longitudinally along the first flexible elongate member;
positioning a stiffening wire in the first groove, wherein the stiffening wire is coupled to a second flexible elongate member; and
positioning an electrical cable in the second groove, wherein the electrical cable is coupled to a physiologic sensor assembly; and
coupling the first flexible elongate member to the physiologic sensor assembly.

14. The method of claim 13, wherein the first flexible elongate member is tubular in shape, and wherein the first groove and the second groove extend longitudinally along an outer surface of the first flexible elongate member.

15. The method of claim 14, wherein the obtaining includes molding the first flexible elongate member to integrally form the first groove and the second groove on the outer surface of the first flexible elongate member.

16. The method of claim 14, wherein the obtaining includes removing material from a blank of the first flexible elongate member to form the first groove and the second groove on the outer surface of the first flexible elongate member.

17. The method of claim 13, wherein the first flexible elongate member includes:
a first distal end;
a first proximal end;
a first inner member portion at the first distal end; and
a second inner member portion adjacent to the first inner member portion,
wherein the method further comprises positioning the first flexible elongate member in a first lumen of a third flexible elongate member sized and shaped for insertion into a vessel of a patient,
wherein the third flexible elongate member includes a second distal end and a second proximal end,
wherein the first flexible elongate member is positioned along a longitudinal axis of the third flexible elongate member such that the first inner member portion extends beyond the second distal end of the third flexible elongate member, and
wherein the first flexible elongate member is a distal inner member, the second flexible elongate member is a proximal outer member, and the third flexible elongate member is a distal outer member.

18. The method of claim 17, further comprising:
reducing an outer diameter of the first inner member portion based on an inner diameter of a second lumen of the physiologic sensor assembly; and wherein
coupling the first flexible elongate member to the physiologic sensor assembly includes positioning the first flexible elongate member within the physiologic sensor assembly such that the first inner member portion extends through the second lumen of the physiologic sensor assembly and distally beyond the physiologic sensor assembly.

19. The method of claim 17, further comprising coupling a distal tip member to the first distal end of the first flexible elongate member after coupling the first flexible elongate member to the physiologic sensor assembly.

20. The method of claim 17, wherein the first flexible elongate member further includes a third inner member portion adjacent to the second inner member portion, and wherein the method further comprises:
coupling the second flexible elongate member to the second proximal end of the third flexible elongate member; and
creating a guide wire exit port at a coupling junction between the second flexible elongate member and the third flexible elongate member during the coupling such that the third inner member portion is in communication with the guide wire exit port.
